(19) United States
(12) Patent Application Publication
(10) Pub. No.: US 2012/0142605 A1
(43) Pub. Date: Jun. 7, 2012

(54) METHODS OF TREATING PSORIASIS

(75) Inventors: Michele M. Hooper, Oak Park, CA (US); Elizabeth H.Z. Thompson, Newbury Park, CA (US)

(73) Assignee: Amgen Inc., Thousand Oaks, CA (US)

(21) Appl. No.: 13/319,004

(22) PCT Filed: May 5, 2010
(86) PCT No.: PCT/US10/33775

§ 371 Date: Feb. 21, 2012

Related U.S. Application Data
(60) Provisional application No. 61/175,748, filed on May 5, 2009.

(51) Int. Cl. A61K 38/16; A61P 17/06

(52) U.S. Cl. 514/18.6

(57) ABSTRACT

The invention includes methods for treating patients having psoriasis and methods for testing the efficacy of such treatments. The methods include treating the patients with a TNF inhibitor plus a topical preparation containing a glucocorticoid.

### CROSS REFERENCE TO RELATED APPLICATIONS

This application claims the benefit under 35 U.S.C. 119(e) of U.S. patent application No. 61/175,748, filed May 5, 2009, which is incorporated herein by reference.

### FIELD

This invention is in the field of methods for treating psoriasis and methods for determining the efficacy of a psoriasis treatment.

### BACKGROUND OF THE INVENTION

Psoriasis is a very common chronic inflammatory disease of the skin that occurs in 2-3% of the world population. Kormeili et al. (2004), British J. Dermatol. 151: 3-15. It is most usually a chronic, life-long condition and can have profound effects on the health and well-being of patients. Nast et al. (2007), Arch. Dermatol. Res. 299: 111-38. Patients can experience periods of increased disease activity even when being treated. Menter et al. (2008), J. Amer. Acad. Dermatol. 58: 826-50, 837-38.

Various topical and systemic treatments are currently used for the treatment of psoriasis. Topical treatments are usually the treatments of first choice for patients with localized disease. Menter et al. (2009), J. Amer. Acad. Dermatol. 60: 643-59, 644. Such topical treatments include topical preparations containing glucocorticoids, which can be highly effective. Id., at 645-46. However, topical glucocorticoids can be used only for limited periods of time, since their use can be accompanied by serious toxicities. Id., at 646. Psoriasis patients are not generally considered to be candidates for systemic treatment unless topical treatments, such as topical glucocorticoids, among others, have already failed to control disease symptoms or the toxicities of such topical treatments cannot be tolerated by the patient. Other factors may also influence this decision such as the extent of the body surface covered by lesions or the existence of particularly recalcitrant lesions.

A number of drugs that inhibit TNF-α have been approved for the treatment of psoriasis, including the biologics infliximab (REMICADE®, Centocor, Inc., Horsham, Pa., U.S.A.), adalimumab (HUMIRA®, Abbott Laboratories, Abbott Park, Ill., U.S.A.), and etanercept (ENBREL®, Amgen Inc., Thousand Oaks, Calif., U.S.A.). Inflixmab and adalimumab are, respectively, a chimeric antibody and a fully human antibody that bind to human TNF-α. Etanercept is a fusion protein consisting of the extracellular region of the human p75 TNF receptor fused to an Fc portion of an antibody, which is currently in use as a systemic treatment for moderate to severe plaque psoriasis. ENBREL® (Etanercept) Package Insert, 2008. Although etanercept can be safely and effectively used for at least 96 weeks in psoriasis patients, it is not completely effective for all patients. Tyring et al. (2007), Arch. Dermatol. 143: 719-26. Thus, there is a need in some patients, to increase its efficacy, especially during periods of increased disease activity.

### SUMMARY

The instant invention encompasses methods for treating patients having psoriasis, which include administering a TNF inhibitor, for example etanercept, plus a topical preparation containing a glucocorticoid. Optionally, the patients have plaque psoriasis. The invention includes methods for determining the efficacy of such a treatment.

In one embodiment, the invention encompasses a method for treating a patient having psoriasis, comprising the following steps: (a) selecting a patient who has a PASI score of at least about 10 and at least about 10% of his/her body surface area (BSA) affected by psoriasis and/or a sPGA score of >2 or ≧3; (b) administering a TNF inhibitor, for example etanercept, to the patient at a dose of about 50 mg twice per week for a first time period of about 12 weeks and then administering the TNF inhibitor to the patient at a dose of about 50 mg per week for an additional time period of about 12 weeks; (c) administering an upper midstrength, potent, or superpotent topical preparation containing a glucocorticoid in any one of Classes 1-3 to the patient once during the first and/or the additional time period of (b), wherein the topical glucocorticoid is administered at least once per week for a continuous time period of not more than four weeks, or administering the topical preparation intermittently during the first and/or additional 12 week period of (b), wherein the topical preparation is administered on an as-needed basis for one or more continuous time periods of not more than four weeks each, wherein each continuous time period in which the topical preparation is administered is separated by an intervening continuous time period in which the topical preparation is not administered that is at least as long as the preceding continuous time period during which the topical preparation was administered; and (d) assessing the PASI score and/or the sPGA score of the patient at the end of the first time period of (b); wherein the treatment is therapeutically effective. The patient can achieve a PASI 50, PASI 75, or PASI 90 by the end of the first and/or the additional time period of (b). The patient can have a sPGA score of clear or almost clear at the end of the first and/or the additional time period of (b). The TNF inhibitor administration can be continued for at least about six months or at least about a year following the additional time period of (b) at a dose of about 50 mg per week, and the patient can maintain the PASI 50, PASI 75, or PASI 90 at the end of the six months or at the end of the year. The topical preparation can be administered during one continuous time period or during intermittent continuous time periods during the year following the additional time period of (b), wherein each continuous time period in which the topical preparation is administered is not more than two, three, four, five six, seven, or eight weeks, and wherein each continuous time period in which the topical preparation is administered is separated by an intervening continuous time period in which the topical preparation is not administered at least as long as the preceding continuous time period during which the topical preparation was administered. The topical preparation can be administered during not more than three, four, five, six, seven, eight, nine, ten, eleven or twelve continuous time periods during the six months following the additional time period of (b). The topical preparation can be administered during not more than three, four, five, six, seven, eight, nine, ten, eleven, twelve, thirteen, fourteen, fifteen, sixteen, seventeen, eighteen, nineteen, twenty, twenty one, twenty two, twenty three, or twenty four continuous time periods during the year following the additional time period of (b). The glucococorticoid in the topical preparation can betamethasone dipropionate or clobetasol propionate, and the topical preparation can also contain calcipotriene.

The invention includes a method for testing the efficacy of a psoriasis treatment comprising registering a group of patients suffering from plaque psoriasis covering at least about 10% of the body surface area and having a PASI score of at least about 10 and/or having a sPGA score of >2 or ≧3 for a clinical trial, wherein the group of patients comprises at least about 100 patients; separating the patients into two groups; recording the PASI and/or sPGA scores of the patients in the two groups; providing the first of the two groups of patients with a TNF inhibitor, for example etanercept, and an upper midstrength, potent, or superpotent topical preparation containing a glucocorticoid of any one of Classes 1-3 and providing the second of the two groups of patients with the TNF inhibitor alone or with a placebo topical preparation, wherein the TNF inhibitor and the topical preparation containing the glucocorticoid or the placebo topical preparation are administered to the patients; recording the PASI score and/or the sPGA score of the first and second groups of patients after about 12 weeks of administration of the TNF inhibitor and the topical preparation containing the glucocorticoid or the placebo topical preparation, and determining the proportion of patients in the first and second groups that achieve at least a PASI 75. In some embodiments, at least about 50%, 60%, 61%, 70%, or 80% of the patients in the first group achieve a PASI 50, PASI 75, and/or PASI 90 after about 12 and/or after 24 weeks of treatment. The glucocorticoid can be clobetasol propionate or betamethasone dipropionate. The topical preparation containing the glucocorticoid and the placebo topical preparation may be administered for no more than two continuous two, three, four, five, six, seven, or eight week periods separated by at least about two, three, four, five, six, seven, or eight weeks on an as needed basis during the twelve weeks of treatment with the TNF inhibitor.

In another embodiment, the invention encompasses a method for determining the efficacy of a psoriasis treatment comprising registering a group of patients suffering from plaque psoriasis covering at least about 10% of the body surface area and having a PASI score of at least about 10 and/or having a sPGA score of >2 or ≧3 for a clinical trial, wherein the group of patients comprises at least about 100 patients; separating the patients into two groups; recording the PASI and/or sPGA scores of the patients in the two groups; providing the first of the two groups of patients with a TNF inhibitor, for example etanercept, and an upper midstrength, potent, or superpotent topical preparation containing a glucocorticoid and a second group of patients with the TNF inhibitor and a placebo topical preparation, wherein the TNF inhibitor and the topical preparation containing a glucocorticoid or placebo topical preparation are given to the patients for the purpose of self-administration; recording the PASI score and/or the sPGA score of the first and second groups of patients after about 12 and/or 24 weeks of administration of the TNF inhibitor and the topical preparation containing a glucocorticoid or topical placebo preparation, wherein at least about 50%, 60%, 70%, or 80% of the patients in the first group have achieved a PASI 50, PASI 75, and/or PASI 90 after 12 weeks of treatment. The glucocorticoid can be clobetasol propionate or betamethasone dipropionate.

In another aspect, the invention includes a method for treating a psoriasis patient who is receiving a TNF inhibitor, for example etanercept, comprising continuing treatment with the TNF inhibitor, initiating treatment with an upper midstrength, potent, or superpotent topical preparation containing a glucocorticoid, continuing treatment with the topical preparation containing the glucocorticoid for a first time period of not more than two, three, four, five, six, seven, or eight weeks, and then discontinuing treatment with the topical preparation containing the glucocorticoid for a second time period of at least as long as the first time period. The patient can continue the treatment with the TNF inhibitor for at least about a year and can maintain a sPGA score of 0 or 1 or an sPGA of 0, 1, or 2 for the year during which treatment is continued. The glucocorticoid can be clobetasol propionate or betamethasone dipropionate.

In still another aspect, the invention encompasses a method for treating a patient having psoriasis and having a PASI score of at least about 10 and an affected BSA of at least about 10% and/or a PGA score of >2 or ≧3 comprising administering to the patient a TNF inhibitor, for example etanercept, at a dosage of about 50 mg twice per week for at least about 12 weeks, administering to the patient a topical preparation comprising clobetasol proprionate or betamethasone diproprionate plus calcipotriene as needed for at most two, three, four, five, six, seven, or eight weeks during the twelve weeks, wherein the patient achieves a PASI 75 after about 12 weeks of treatment; administering the TNF inhibitor for an additional time period of at least about 12 weeks thereafter at a dosage of about 50 mg per week, wherein the patient maintains a PASI 75 at the end of the additional time period.

In another aspect, the invention comptemplates a method for treating psoriasis comprising: (a) selecting a patient who has a sPGA score of >2 or ≧3 and/or a PASI score of at least 10 with at least 10% of the BSA affected, wherein the patient does not have a condition selected from the group consisting of congestive heart failure, severe pulmonary disease, systemic lupus erythematosus, multiple sclerosis, or any other demyelinating disease, pregnancy, and breast feeding, wherein the patient does not exhibit hemoglobin<11 g/dL, platelet count<125,000/mm³, white blood cell count<3000 cells/mm³, AST and/or ALT≧1.5× the upper limit of normal, and wherein a topical preparation containing glucocorticoid has previously been used to treat the patient's psoriasis, wherein the patient did not achieve a sPGA of clear or almost clear during this treatment; (b) administering to the patient a TNF inhibitor, for example etanercept; and (c) intermittently administering to the patient an upper midstrength, potent, or superpotent topical preparation containing a glucocorticoid on an as-needed basis in order to maintain an sPGA score of clear or almost clear.

### BRIEF DESCRIPTION OF THE FIGURES

FIG. 1 shows a diagram of the timeline and treatments to be included in a clinical trial. “BIW” means twice per week, and “QW” means once per week.

FIG. 2 shows a diagram of the time line and treatments to be included in a clinical trial. “BIW” and “QW” have the same meanings as in FIG. 1.

### DETAILED DESCRIPTION

The invention includes methods for treating psoriasis and methods for determining the efficacy of a treatment of psoriasis. One method for treating psoriasis encompassed by the invention comprises administering to a psoriasis patient a systemic TNFα inhibitor plus a topical preparation containing a glucocorticoid. The TNFα inhibitors contemplated include etanercept or an anti-TNFα antibody, such as, for example, adalimumab, infliximab, certolizumab pegol (marketed as CIMZIA®, UCB S.A., Brussels, Belgium), or golimumab (SIMPONI™, Centocor, Inc. Horsham, Pa., U.S.A.). The topical preparations containing glucocorticoids contemplated include preparations containing a a wide variety of glucocorticoids as described below, which include clobetasol or betamethasone, optionally plus a vitamin D analog, such as calcipotriene. In another embodiment, the invention encompasses methods for determining the efficacy of such a combination therapy including (1) selecting an appropriate group of psoriasis patients, (2) observing the disease status of the patients at baseline, (3) providing the patients with a combination therapy including a TNF-α inhibitor plus a topical preparation containing a glucocorticoid, and (4) observing that at least a certain percentage of patients have achieved a particular disease status or improvement in disease status after a certain length of treatment, such as, for example, about 2, 3, 4, 6, 8, 10, 12, or 24 weeks of treatment.

The term “glucocorticoid,” as meant herein, is described in detail below. The term “corticosteroid” refers to a group of hormones secreted by the adrenal cortex and analogues thereof. “Corticosteroid” encompasses glucocorticoids, mineralocorticoids, such as aldosterone, and sex steroids. The glucocorticoids, as typified by cortisol, bind to glucocorticoid receptors and have anti-inflammatory, metabolic, and immunoregulatory effects and regulate key homeostatic functions. Some corticosteroids, such as, for example, cortisone, have both glucocorticoid and mineralocorticoid effects, while others, have primarily glucocorticoid effects.

A “TNF inhibitor,” as meant herein is any molecule that can inhibit the activity of human TNF-α, such as, for example, etanercept, infliximbab, adalimumab, golimumab, or certolizumab. Small molecules can also be TNF-α inhibitors. The activity of TNF can be measured by the L929 cytolysis assay as described by Mohler et al. (1993), J. Immunol. 151: 1548-61, the portion of which describes this assay is incorporated herein by reference.

As meant herein, “alkyl” or the prefix “alk-” denotes an alkyl group having from one to six carbons.

As meant herein, “halogen” or the prefix “halo-” denotes a fluorine, chlorine, bromine, or iodine.

As meant herein, “haloalkoxy” describes groups such as —O—CH₂—F, —O—CH₂—Br, or other similarly structured haloalkoxy groups that have different halogen or alkyl groups.

As meant herein, “halothioalkyl” describes molecules such as —S—CH₂—F, —S—CH₂—Br, or other similarly structured halothioalkyl groups that have different halogen or alkyl groups.

As meant herein, a “acetyl” group has the following structure:

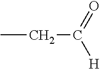

As meant herein, a “propionyl” group has the following structure:

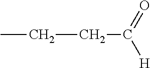

As meant herein, a “butyryl” group has the following structure:

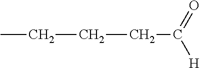

As meant herein, a “valeryl” group has the following structure:

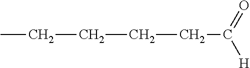

Measurement of the levels of alanine amino transferase (ALT) and aspartate aminotransferase (AST) can provide an indication of liver, muscle, or heart damage. Levels of ALT and AST can be measured with routine blood tests. The upper limit for a normal ALT score is considered herein to be 500 nkat/L (36 U/L) for men and 317 nkat/L (21 U/L) for women. The upper limit of normal for a normal AST score is considered to be 40 U/L for men and 35 U/L for women.

The methods of the invention utilize a topical preparation containing a glucocorticoid. In some embodiments, a potent or a superpotent preparation of a topical glucocorticoid is used. The glucocorticoids of the invention encompass molecules having the structure and formula shown below.

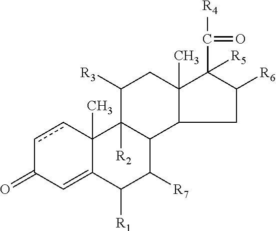

R1 can be a hydrogen, an alkyl, or a halogen. R2 can be a hydrogen or a halogen. R3 can be a carbonyl oxygen or a hydroxyl group. R4 can be alkyl, hydroxyalkyl, optionally hydroxymethyl, haloalkyl (e.g., —CH₂—F or —CH₂—Cl), haloalkoxy (e.g. —O—CH₂—F), a halothioalkyl (e.g., —S—CH₂—F), alkoxy, —CH₂—O-acetyl, or —CH₂—O-propionyl. R7 can be a hydrogen or a halogen. R5 can be a hydroxyl group or an —O-acetyl, —O-propionyl, O-butyryl, O-valeryl, or alkoxycarbonyloxy (e.g. an ethoxycarbonyloxy) group. R6 can be a hydrogen, or an alkyl (e.g., a methyl), methylene, or hydroxyl group. Alternatively, R5 and R6 may include oxygen atoms that are both covalently bound to an additional carbon atom, which forms part of one of the structures shown below:

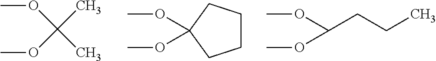

In addition, these glucocorticoids can be, for example, adamantoate, diacetate, benzoate, propionate, dipropionate, valerate, acetate, hexanoate, aceponate, pivalate, butyrate, succinate sodium, or phosphate disodium salts of the above compounds.

“Topical preparations containing glucocorticoids” can be, for example, in the form of an ointment, a powder, a gel ointment, an emollient, a cream, an adhesive patch or strip, a shampoo, a spray, a foam, a gel, or a liquid.

In addition to the “glucocorticoids” as described by the chemical formula above, “glucocorticoids,” as meant herein, include the following specific glucocorticoids, a few of which are outside the ambit of the chemical formula above: 21-acetoxypregnenolone, alclometasone (for example, alclometasone dipropionate (such as the cream or ointment sold under the trade name ACLOVATE®, GlaxoSmithKline)), algestone, amcinonide, beclomethasone (for example, beclomethasone dipropionate), betamethasone (for example, betamethasone valerate, for example, LUXIQ® foam, which is 0.12% betamethasone valerate, or betamethasone diproprionate, optionally plus calcipotriene, such as the product sold under the trade name TACLONEX® by Warner Chilcott), budesonide, clobetasol (for example, clobetasol propionate, including the spray, lotion, or shampoo sold by Galderma under the tradename CLOBEX®, the ointment, cream, or gel sold by GlaxoSmithKline under the trade name TEMOVATE® or TEMOVATE E®, or the 0.05% clobetasol propionate foam sold under the tradename OLUX® or OLUX-E®), clobetasone (for example, clobetasone butyrate), clobetasol propionate, clocortolone (for example, clocortolone acetate or clocortolone pivalate), cloprednol, corticosterone, cortisone, cortivasol, deflazacort, desonide, desoximetasone (for example, desoximetasone acetate), 11-desoxy-17-hydroxycorticosterone, dexamethasone, diflorasone (for example, diflorasone acetate or diflorasone diacetate), diflucortolone (for example, diflucortolone valerate), difluprednate, enoxolone, fluazacort, flumethasone (including flumethasone acetate and flumethasone pivalate), fluchloronide, flunisolide, fluorometholone (for example, fluorometholone acetate), fluocinolone (for example, fluocinolone acetonide), fluocinonide, fluocortolone (including fluocortolone acetate, fluocortolone hexanoate, and fluocortolone pivalate), fluocortin butyl, fluperolone (including fluperolone acetate), fluprednidene (including fluprednidene acetate), fluprednisolone (including fluprednisolone acetate), flurandrenolide (including flurandrenolide acetonide), fluticasone (for example, fluticasone propionate), formocortal, halcinonide (which includes halcinonide desoximetasone and preparations sold under the tradenames HALOG® and HALOG E®), halobetasol (for example, halobetasol propionate, such as the cream or ointment sold by Bristol-Myers Squibb under the trade name ULTRAVATE®), halometasone, halopredone acetate, hydrocortamate, hydrocortisone (for example, hydrocortisone acetate, hydrocortisone butyrate, hydrocortisone probutate, hydrocortisone phosphate, hydrocortisone succinate sodium, or hydrocortisone valerate, and including the cream sold under the trade name ANUSOL-HC® 2.5% by Monarch Pharmaceuticals), mazipredone, methylprednisolone, mometasone, including mometasone furoate, paramethasone (for example, disodium phosphate), medrysone, meprednisone (for example, meprednisone acetate), methylprednisolone (for example methylprednisolone acetate, disodium phosphate, sodium succinate, or aceponate), prednicarbate, prednisolone, prednisone, prednival, prednylidene (for example, prednylidene diethylaminoacetate), tixocortol (for example, tixocortol pivalate), or triamcinolone (for example triamcinolone acetonide).

Topical preparations containing glucocorticoids may include an active ingredient other than the glucocorticoid, for example a vitamin D compound or an analog thereof, such as calcipotriene or calcipotriol. One example of such a product is TACLONEX®, which contains calcipotriene (0.005%) and betamethasone dipropionate (0.064%).

The glucocorticoid in a topical preparation can be, for example, at a concentration of 0.0005%, 0.01%, 0.02%, 0.025%, 0.03%, 0.04%, 0.05%, 0.1%, 0.25% 0.5%, 1.0%, 1.5%, 2.0%, 2.5%, or 3.0%, or approximately at these concentrations. Additionally, the concentration of the glucocorticoid can be from 0.0005% to 0.05%, from 0.01% to 5.0%, from 0.03% to 0.08%, from 0.05% to 1.0%, from 1.0% to 2.0%, from 2.2% to 2.8%, from 2.0% to 3.0%, or from 3.0% to 4.0%, or within approximately these ranges.

The potency of a topical preparation containing a glucocorticoid is typically classified with respect to potency by the vasoconstrictor assay. Hengge et al. (2006), J. Am. Acad. Dermatol. 54: 1-15, at 2; Stoughton, “Vasoconstrictor Assay—Specific Applications,” in Topical Corticosteroids, Maibach and Surbur, eds., Karger, Basel, 1992, pp. 42-53; Cornell and Stoughton (1985), Arch. Dermatol. 121: 63-67. This assay can be performed as follows. A group of normal human subjects is selected that is sufficient in number for the number of compounds to be tested. For example, about thirty subjects are sufficient to evaluate eight different formulations. A negative control (consisting of an ointment, cream, spray, or other topical vehicle without any glucocorticoid) and a positive control (a formulation of known high potency that contains a known conticosteroid) are used to compare the test samples to. The flexural aspects of the forearms of each subject are cleansed, and about 10 milligrams of test material, or negative or positive control samples, is applied to each of four 3 cm² areas on the flexural aspect of each forearm. Thus, each subject has eight test areas. The area is covered with, an elevated hard plastic guard that has holes in it to allow free air movement, and the samples are left in place for 16 hours after application. Then the plastic guards are removed, and the test sites are gently washed with soap and water. At 18 hours after application, the intensity of vasoconstriction at the test sites is assessed. The vasoconstriction at the test sites is preferably evaluated by one person to eliminate differences in scoring between individuals. Each test site is scored as a 0 (no vasoconstriction), 1 (mild vasoconstriction), 2 (moderate vasoconstriction), or 3 (marked vasoconstriction). Vasoconstriction is visible as a whitening of the skin. The formulations to be tested are coded to eliminate any potential bias on the part of the investigator. The description of this assay by Cornell and Stoughton (1985), Arch. Dermatol. 121: 63-67 is incorporated herein by reference. Formulations have been classified into the following seven groups based on this assay:

Class 1, superpotent; Class 2, potent; Class 3, upper mid-strength; Class 4, midstrength; Class 5, lower midstrength; Class 6, mild; and Class 7, least potent. Table 1 below lists a number of commercial formulations that have been classified as to potency with this assay. See, e.g., Stoughton, “Vasoconstrictor Assay—Specific Applications,” in Topical Corticosteroids, Maibach and Surbur, eds., Karger, Basel, 1992, pp. 42-53; Jacob and Steele (2006), J. Am. Acad. Dermatol. 54(4): 723-27. Other formulations with activity in the vasoconstrictor assay comparable to preparations listed in Table 1 in the vasoconstriction assay will be classified in the same classes.

TABLE 1
Potency ranking of exemplary topical preparations
containing a glucocorticoid

| | |
|---|---|
| Brand Name | Generic Name (concentration) |

Class 1: Superpotent

| | |
|---|---|
| TEMOVATE ® cream or ointment | clobetasol propionate (0.05%) |
| CLOBEX ® spray, lotion, or | clobetasol propionate (0.05%) |
| shampoo | |
| OLUX ® foam | clobetasol propionate (0.05%) |
| DIPROLENE ® cream or | betamethasone dipropionate (0.05%) |
| ointment | |
| PSORCON ® ointment | diflorasone diacetate (0.05%) |
| ULTRAVATE ® cream or | halobetasol propionate (0.05%) |
| ointment | |

Class 2: Potent

| | |
|---|---|
| CYCLOCORT ® ointment | amcinonide (0.1%) |
| DIPROLENE ® cream AF | betamethasone dipropionate (0.05%) |
| DIPROSONE ® ointment | betamethasone dipropionate (0.05%) |
| ELOCON ® ointment | mometasone furoate (0.1%) |
| FLORONE ® ointment | diflorasone diacetate (0.05%) |
| HALOG ® cream | halcinonide (0.1%) |
| LIDEX ® cream, gel, or ointment | fluocinonide (0.05%) |
| MAXIFLOR ® ointment | diflorasone diacetate (0.05%) |
| TOPICORT ®cream, gel, or | desoximetasone (0.25%, 0.05%, or |
| ointment | 0.25%) |

Class 3: Upper Midstrength

| | |
|---|---|
| ARISTOCORT A ® ointment | triamcinolone acetonide (0.1%) |
| CYCLOCORT ® cream or lotion | amcinonide (0.1%) |
| DIPROSONE ® cream | betamethasone dipropionate (0.05%) |
| FLORONE ® cream | diflorasone diacetate (0.05%) |
| LIDEX E ® cream | fluocinonide (0.05%) |
| HALOG ® ointment | halcinonide (0.1%) |
| MAXIFLOR ® cream | diflorasone diacetate (0.05%) |
| VALISONE ® ointment | betamethasone valerate (0.1%) |
| CUTIVATE ® ointment | fluticasone propionate (0.005%) |

Class 4: Midstrength

| | |
|---|---|
| ELOCAN ® cream | mometasone furoate (0.1%) |
| KENALOG ® cream | triamcinolone acetonide (0.1%) |
| SYNALAR ® ointment | fluocinolone (0.025%) |
| WESTCORT ® ointment | hydrocortisone valerate (0.2%) |

Class 5: Lower Midstrength

| | |
|---|---|
| CORDRAN ® cream | flurandrenolide (0.05%) |
| DIPROSONE ® lotion | betamethasone dipropionate (0.05%) |
| KENALOG ® lotion | triamcinolone acetonide (0.1%) |
| SYNALAR ® cream | fluocinolone (0.025%) |
| VALISONE ® cream | betamethasone valerate (0.1%) |
| WESTCORT ® cream | hydrocortisone valerate (0.2%) |
| DERMATOP ® cream | prednicarbate (0.1%) |
| LOCOID ® cream | hydrocortisone butyrate (0.1%) |

Class 6: Mild

| | |
|---|---|
| ALCOVATE ® cream or ointment | aclometasone (0.05%) |
| LOCORTEN ® cream | flumethasone pivalate (0.03%) |
| SYNALAR ® solution or cream | fluocinolone (0.01%) |
| TRIDESILONE ® cream | desonide (0.05%) |
| VALISONE ® lotion | betamethasone valerate (0.05%) |

Class 7: Least Potent

various preparations containing
hydrocortisone, dexamethasone,
flumethalone, prednisolone, or
methyprednisolone

The methods of the invention can utilize any topical preparation containing a glucocorticoid. In some embodiments, preparations from one or more of Classes 1-7, as described above, are used in the methods of the invention. For example, a superpotent (Class 1) topical preparation containing a glucocorticoid can be used. In other embodiments, a potent or superpotent topical preparation containing a glucocorticoid preparation is used, where the preparation is from Classes 1 or 2. In other embodiments, a topical preparation containing a glucocorticoid that is a potent, superpotent, or upper midstrength preparation from any of Classes 1-3 is used. In still other embodiments, a lower midstrength, midstrength, upper midstrength, potent, or superpotent topical preparation containing a glucocorticoid from any of Classes 1-5 is used. Alternatively, a midstrength or lower midstrength preparation containing a glucocorticoid from Class 4 or 5 is used.

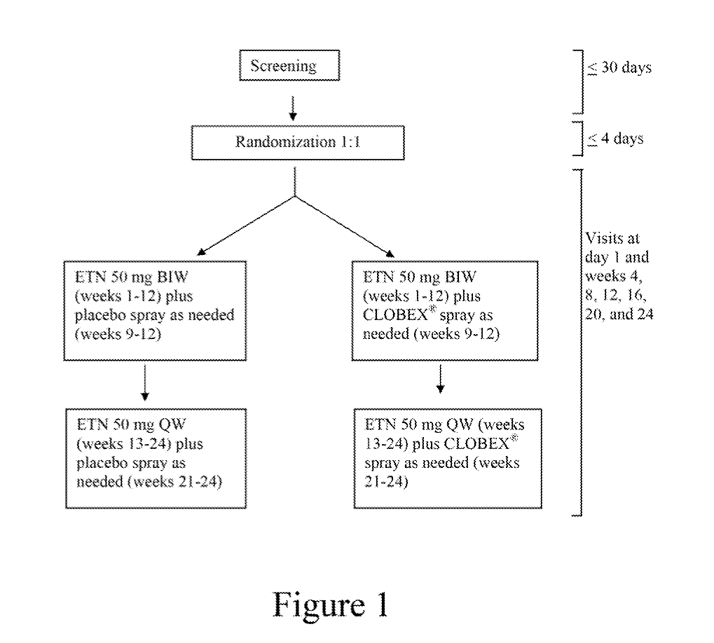

The absorption of glucocorticoids in topical preparations varies significantly between different individuals and between different parts of the body. Hengge et al. (2006), J. Am. Acad. Dermatol. 54: 1-15. For example, while absorption of hydrocortisone applied to the sole of the foot, the ankle, the palm, the forearm, or the back is relatively low, absorption of hydrocortisone applied to the scalp, axilla, forehead, or jaw angle is much higher. Id., FIG. 1, page 3. Absorption from application on the eyelid is about 300 fold greater than absorption from the plantar skin, i.e., the skin on the sole of foot. Children and infants have increased surface to weight ratios as compared to adults, leading to concern about increased absorption as compared to adults. Similarly, the elderly typically have fragile skin, a condition that may also lead to increased systemic absorption. Id., at page 1. It is known, for example, that absorption of glucocorticoids applied to diseased skin of atopic dermatitis patients is 2 to 10 times more effective than absorption through healthy skin. Id., at page 2.

The absorption and potency of a topical preparation containing a glucocorticoid can also be affected by the vehicle in which the glucocorticoid is formulated. Id.; Ayres and Hooper (1978), Br. J. Dermatol. 99: 307-17. A “vehicle,” as meant herein, is the gel, cream, ointment, liquid, foam, spray, or other formulation in which a glucocorticoid is carried. As is evident from the collection of data in Table 1, it is possible for two different topical preparations containing the same glucocorticoid at the same concentration to differ in potency. Such results may be accounted for by differences in the vehicle in which glucocorticoid is carried.

Some ingredients that may be included in the vehicle of a topical glucocorticoid preparation include the following: benzyl alcohol, petrolatum, white petrolatum, stearyl alcohol, propylene glycol, isopropyl myristate, polyoxyl 40 stearate, carbomer 934, carbomer 934P, sodium lauryl sulfate, edentate disodium, sodium hydroxide, glyceryl monolaurate, glyceryl monomyristate, citric acid, cetostearyl alcohol, isopropyl myristate, propylene glycol, cetomacrogol 1000, dimethicone 360, sodium citrate, imidurea, cetearyl alcohol, glyceryl stearate, PEG 100 stearate, Ceteth-20, monobasic sodium phosphate, chlorocresol, phosphoric acid, and water.

The adverse effects of topical application of a glucocorticoid can be local and/or systemic, with local reactions being much more common Id., at page 4. Local adverse reactions include the following: tachyphylaxis, which is decreased efficacy of the glucocorticoid; atrophy of the skin, including increased transparency and shininess of the skin as well as the appearance of striae; telangiectasia, which is characterized by abnormal dilation of capillary vessels and arterioles; disturbance of the epidermal barrier, such as increased transepidermal water loss; appearance of striae, which are visible, linear scars; acne-like lesions; perioral dermatitis, which is composed of follicular papules and pustules on an erthematous background that begin near the mouth; increased growth of vellus hair and, rarely, hypertrichosis or excessive body hair; hypopigmentation; purpura; stellate pseudoscars; ulcerations; aggravation of cutaneous infections; delayed wound healing; contact allergies to glucocorticoids; decreased in skin elasticity; and drying, wrinkling, and loosening of the skin.

Systemic adverse effects of topical application of a glucocorticoid can be serious. Adverse effects associated with the use of topical glucocorticoids include the following: glaucoma following use of a glucocorticoid preparation around the eye; cataract formation; hypothalamic-pituitary-adrenal axis suppression; Cushing syndrome; Addisonian crisis; retarded growth in children; and promotion or exacerbation of hyperglycemia. Such effects may be more frequent with prolonged use, use of more potent preparations, use in particularly sensitive areas (such as around the eyes), and/or use in children or the elderly. Thus, long-term treatment, especially with potent or superpotent topical glucocorticoids, is not desirable, and treatment should be intermittent.

Treatment with a topical preparation containing a glucocorticoid on “as-needed basis,” as meant herein, means that treatment is used only when psoriasis symptoms are present. At the start of treatment psoriasis is present, and the glucocorticoid is administered. If a patient's psoriasis symptoms become minimal, that is, if the patient attains a sPGA score of 0 or 1, treatment with the topical preparation would be discontinued. If psoriasis symptoms re-emerge, treatment would be re-initiated. Administration of a topical glucocorticoid on an “as-needed basis” for a given time period means that the topical treatment is administered during that time period, until the psoriasis symptoms lessen to a minimal level, that is, if the patient has a sPGA score of 0 or 1. If this is the case, the patient may cease treatment with the topical treatment for some or all of the time period and still be administering on an “as-needed basis” during the time period, as meant herein.

Some superpotent glucocorticoids such as clobetasol propionate foam (0.05%; Olux-E®) are indicated for only mild to moderate psoriasis, whereas etanercept is indicated for moderate to severe psoriasis. For example, in a prior etanercept trial, the average subject had an involved body surface area of approximately 30% when starting treatment, indicating a disease severity that would likely be outside of the mild to moderate range. Such patients are, therefore, not candidates for monotherapy using 0.05% clobetasol propionate foam. However, many patients experience a reduction, but not a complete elimination, of psoriasis symptoms with etanercept treatment. Since, superpotent glucocorticoids such as 0.05% clobetasol propionate foam can be very effective in patients with less widespread disease, such a treatment could provide additional benefit to patients whose disease is largely controlled using etanercept therapy. Thus, patients using etanercept therapy could use a topical preparation containing a glucocorticoid, such as, for example, clobetasol propionate foam, intermittently on their remaining lesions to achieve even better control of their psoriasis symptoms.

The methods of treatment of the invention can also be combined with other psoriasis therapies including, for example, methotrexate, cyclosporine, UVA therapy with or without methoxsalen or psoralen, acitretin, anthralin, topical or oral retinoids, a vitamin D, such as vitamin D3, or an analog thereof such as calcipotriene or calcipotriol, fumarates, Dead Sea salts, sunshine, and UVB therapy.

The invention encompasses the use of any TNF inhibitor with any topical preparation containing a glucocorticoid to treat psoriasis, optionally plaque psoriasis. The TNF inhibitor can be a small molecule. The TNF inhibitor can also be a biologic such as etanercept (ENBREL®, marketed by Amgen Inc., Thousand Oaks, Calif.). The amino acid sequence of etanercept is published in Clinical Pharmacology and Therapeutics 66(2): 209 (August, 1999), which is incorporated by reference herein. The TNF inhibitors that can be used in the invention include anti-TNFα antibodies, adalimumab (HUMIRA®, marketed by Abbott), infliximab (REMICADE®, marketed by Centocor, Inc.), golimumab (SIMPONI™, marketed by Centocor, Inc.) and certolizumab pegol (CIMZIA®, marketed by UCB).

The TNF inhibitors can be administered in a variety of ways. TNF inhibitors that comprise proteins will often be injected, whereas TNF inhibitors that are small molecules may be taken orally or, for example, implanted or inhaled. If injected, a TNF inhibitor can be administered, for example, via intra-articular, intravenous, intramuscular, intralesional, intraperitoneal or subcutaneous routes by bolus injection or by continuous infusion. Other suitable means of administration include sustained release from implants, aerosol inhalation, eyedrops, oral preparations, including pills, syrups, lozenges or chewing gum, and topical preparations such as lotions, gels, sprays, ointments or other suitable techniques.

Appropriate dosages for a TNF inhibitor, optionally a TNF inhibitor comprising a protein, include the following dosages (and dosages that are about the same as these dosages): 25 mg twice per week (BIW); 50 mg once per week (QW); 50 mg BIW; 0.5, 0.8, 1, 2, 3, 4, 5, 6, 7, 8, 9, or 10 mg/kg every 1, 2, 3, 4, 5, 6, 7, 8, 9, or 10 weeks; or 10, 20, 30, 40, 50, 75, 100, 200, 300, 400, or 500 mg every 1, 2, 3, 4, or 5 weeks.

The severity of disease in psoriasis patients is measured in a variety of ways. One way disease activity is commonly measured in clinical trials is the Psoriasis Area and Severity Index, which is commonly referred to as the PASI score. A PASI score can range from 0 to 72, with 72 being the most severe disease. For purposes of PASI assessment, the body is considered to consist of four sections, legs, torso (that is, stomach, chest, back, etc.), arms, and head, which are considered to have 40%, 30%, 20%, and 10% of a person's skin, respectively. For each section, the percent of the area of skin affected is estimated and transformed into a grade of from 0 to 6, with 0 being no affected skin and 6 being 90-100% of the skin of the body section in question being affected. The severity of disease is scored by separately considering three features of the affected skin, redness (erythema), scaling, and thickness, and assigning a severity score of from 0 to 4 for each feature for each body section. The sum of the severity scores for all three features for each body section is calculated, and this sum is multiplied by the weight of the respective section as determined by how much of the total skin that body section contains and by the percent of the body section affected. After this number is calculated for each body section, these numbers are added to yield the PASI score. Thus, the PASI score can be expressed as follows:

$$PASI = 0.1 ( score for percent of the head affected ) ( sum of 3 severity scores for the head ) + 0.2 ( score for percent of the arms affected ) ( sum of 3 severity scores for the arms ) + 0.3 ( score for percent of the torso affected ) ( sum of 3 severity scores for the torso ) + 0.4 ( score for percent of the legs affected ) ( sum of 3 severity scores for the legs )$$

The descriptions of PASI scores in the following two references are incorporated by reference herein: Feldman and Krueger (2005), Ann. Rheum. Dis. 64: 65-68, Langley and Ellis (2004), J. Am. Acad. Dermatol. 51(4): 563-69.

Many clinical trials refer to changes in PASI score over the course of the study. For example, a PASI 75 at a particular time point in a clinical trial means that the PASI score of a patient has decreased by 75% as compared to that patient's PASI score at baseline. Similarly a PASI 50 or a PASI 90 denotes a 50% or 90% reduction in PASI score.

Another commonly used measure of psoriasis severity in clinical trials is the static Physicians Global Assessment (sPGA). The sPGA is typically a six category scale rating ranging from 0=none to 5=severe. ENBREL® (etanercept), Package Insert, 2008. A sPGA score of “clear” or “minimal” (sometimes alternately referred to as “almost clear”) requires no or minimal elevation of plaques, no or only very faint redness, and no scaling or minimal scaling over <5% of the area of the plaques. ENBREL® (etanercept), Package Insert, 2008. The individual elements of psoriasis plaque morphology or degree of body surface area involvement are not quantified. Nonetheless, sPGA scores correlate to some extent with PASI scores. Langley and Ellis (2004), J. Am. Acad. Dermatol. 51(4): 563-69.

Other measures of psoriasis severity take into account a patient's perception of the impact of their psoriasis on their quality of life. One such measure is the Dermatology Life Quality Index (DLQI). This instrument consists of 10 questions. ENBREL® (etanercept), Package Insert, 2008.

A “therapeutically effective” treatment, as meant herein, is a treatment in which a decrease over baseline of at least 1 in a sPGA score or at least 50% in PASI score is observed in at least 20% of the patients treated after about 12 weeks of treatment.

The following example is offered by way of illustration and not limitation. All references cited herein, both supra and infra, are incorporated herein by reference.

### Example 1

The study described below will compare (1) the safety and efficacy of systemic etanercept therapy combined with a topical spray containing clobetasol propionate (0.05%; CLOBEX® spray) with (2) the safety and efficacy of systemic etanercept monotherapy in patients with moderate to severe plaque psoriasis. Efficacy will be measured in a number of ways including the following: 1) the number of patients achieving a PASI 50, PASI 75, and PASI 90 at weeks 12 and 24; 2) the number of patients achieving a sPGA of clear or almost clear at weeks 12 and 24; 3) patient satisfaction at weeks 12 and 24; 4) patient assessment of itch at weeks 12 and 24; and 5) improvement of PASI score from baseline at weeks 12 and 24. Safety will be evaluated by adverse events and by patient laboratory profiles.

Study Design:

This is a randomized, double-blind, placebo-controlled study to evaluate the safety and efficacy of combining short courses of topical spray containing clobetasol propionate with systemic etanercept therapy compared with the safety and efficacy of etanercept monotherapy in patients with moderate to severe plaque psoriasis.

After completing all screening assessments and meeting all eligibility criteria, subjects will be randomized in a 1:1 ratio to receive etanercept 50 mg twice per week (BIW) combined with short courses of a topical spray containing clobetasol propionate (the active arm) or etanercept 50 mg BIW combined with a topical placebo spray (the placebo arm). For the following 12 week period, subjects will receive 50 mg of etanercept per week and either a short course of topical spray containing clobetasol propionate (active arm) or a short course of topical placebo spray (placebo arm). The study design is illustrated by FIG. 1.

The primary efficacy endpoint of the study is the proportion of subjects with a PASI 75 at week 12. Secondary endpoints include the following: the number of subjects with a sPGA score of clear or almost clear at weeks 12 and 24; the number of subjects with a PASI 50, 75, and 90 at weeks 12 and 24; patient satisfaction with treatment (scored as very dissatisfied, dissatisfied, neither satisfied nor dissatisfied, satisfied, or very satisfied) at weeks 12 and 24; patient assessments of pain and itch at weeks 12 and 24; improvement in PASI from baseline at weeks 12 and 24. Adverse events, changes in laboratory profiles, and vital signs will also be monitored.

The inclusion and exclusion criteria shown in Tables 2 and 3 below will be applied in screening patients for the study.

TABLE 2
Inclusion Criteria

Inclusion Criteria
Subject is ≧18 years of age at time of screening.
Subject is capable of understanding and giving written, voluntary informed consent before study
screening.
Subject has had stable moderate to severe plaque psoriasis for at least 6 months.
Subject has psoriatic lesions on ≧10% of his/her body surface area (BSA) and a Psoriasis Area and
Severity Index (PASI) ≧10 at screening and at baseline.
Subject is a candidate for systemic therapy or phototherapy in the opinion of the investigator.
Subject has a negative test for hepatitis B surface antigen and hepatitis C antibody.
Subject has a negative tuberculin skin test within 30 days prior to the first dose of the study drug.
(Tuberculin skin tests should be considered positive when they have greater than or equal to 5 mm of
induration at 48-72 hours after the test is placed. Subjects with a positive tuberculin skin test are
allowed if(1) they have completed treatment with appropriate chemoprophylaxis or (2) they have a
history of Bacillus Calmette-Guerin vaccination with a negative Quantiferon test in the past year, no
symptoms per
tuberculosis worksheet, and a negative chest X-ray.)
All female subjects, except those who are surgically sterile or at least three years post menopause,
have a
negative serum pregnancy test within 28 days before start of trial and willingness to use medically
acceptable birth control for the duration of the study.
Subject or designee is capable of injecting the investigational product subcutaneously.

TABLE 3
Exclusion Criteria

Exclusion Criteria
Subject has active guttate, erythrodermic, or pustular psoriasis at the time of screening.
Subject has skin conditions that would interfere with evaluation of the investigational product on
psoriasis at the time of screening.
Subject has any active infection of CTC grade 2 or higher (including chronic or localized infections)
within 30 days prior to screening, at screening, or during screening period prior to first dose of the
trial.
Subject is known to be HIV positive.
Subject has significant concurrent medical conditions at or during screening, including: type 1
diabetes; poorly controlled type 2 diabetes (HgA1c >8.5); symptomatic heart failure (NYHA class II,
III, or IV); myocardial infarction within the last year; current history of unstable angina pectoris;
uncontrolled hypertension as defined by resting blood pressure >160/90 mmHg assessed on two
separate occasions during the screening period; severe pulmonary disease (requiring hospitalization
during the last year or oxygen therapy); major chronic inflammatory disease or connective tissue
disease other than psoriasis and/or psoriatic arthritis; multiple sclerosis or any other demyelinating
disease; active malignancy, including evidence of cutaneous basal or squamous cell carcinoma or
melanoma; history of cancer (other than fully resected and surgically cured cutaneous basal cell or
squamous cell carcinoma) within five years before the of the trial (documentation of disease-free state
since treatment is required if malignancy occurred more than five years ago); known
immunodeficiency syndromes; and any condition that, in the opinion of the investigator, might cause
this study to be detrimental to the subject.
Subject is pregnant or breast feeding.
Subject is hypersensitive to clobetasol propionate, to other glucocorticoids, or to any ingredient in the
CLOBEX ® preparation.
Subject has used ultraviolet light B (UVB) therapy, topical cyclosporine, calcineurin inhibitors,
topical vitamin A or D analog preparations, or topical steroids of Class 3-7 within 14 days of the first
dosing of the study.
Subject has used one of the following therapies within 28 days of the initiation of dosing in the trial:
intravenous (IV) or oral calcineurin inhibitors; ultraviolet light A (UVA) therapy; psoralen plus
ultraviolet A radiation (PUVA); oral retinoids; Class 1 or 2 topical steroids; anthralin; any other
systemic psoriasis therapy (e.g., methotrexate, cyclosporine), including oral or parenteral
corticosteroids; cyclophosphamide; or sulfasalazine; or anakinra.
Subject has used a biologic therapy, other than (IL)-12/IL-23 inhibitors or anti-TNF agents, within
three months of the start of dosing in the trial.
Subject has used an interleukin (IL)-12/IL-23 inhibitor within 6 months of the start of the trial.
Subject has used an anti-TNF agent within three months of the start of the trial. Subjects with prior
use of an anti-TNF agent more than three months before the start of the trial must be excluded if they
have (1) discontinued use of an anti-TNF agent for a reason other than lack of efficacy or (2)
experienced a clinically significant adverse event (such as a serious infection, neurological event,
malignancy, hematologic event, or any other adverse event that the investigator feels might cause this
study to be detrimental to the subject).
Subject has laboratory abnormalities at screening or baseline, including: hemoglobin <11 g/dL;
platelet count <125,000/mm³; white blood cell count < 3,500 cells/mm³; AST and/or ALT ≧1.5 ×
the upper limit of normal; creatinine clearance <50 mL/min (Cockroft-Gault formula); any other
laboratory abnormality, which, in the opinion of the investigator, will prevent the subject from
completing the study or will interfere with the interpretation of the study results.
Subject has used a topical preparation containing a glucorticoid used in the trial more recently than 30
days or five half-lives of the glucocorticoid before the start of the trial, whichever is longer.

Patients will be randomized 1:1 to receive (1) etanercept and topical spray containing clobetasol propionate (active arm) or (2) etanercept and a topical placebo spray (placebo arm), as diagrammed in FIG. 1. Etanercept dosing in both arms will be as follows: 50 mg twice weekly for 12 weeks followed by 50 mg per week for the following 12 weeks. No topical treatment will be used in either arm during weeks 1-8 and 13-20 of the trial. Clobetasol propionate spray (active arm) or a placebo spray (placebo arm) will be used as needed during weeks 9-12 and 21-24. Use of clobetasol propionate spray will be limited to (1) treatment of no more than 20% BSA, (2) treatment duration of no more than 4 consecutive weeks, (3) use for the minimum time necessary to achieve desired results; (4) use of no more than 50 g per week, and (5) use not allowed on the face, groin, or axillae.

Patients will be assessed for various medical parameters throughout the Investigational Protocol (IP) as detailed in Table 4. The efficacy evaluable subset will include intent to treat (ITT) set with all randomized subjects, with the analysis conducted according to the original randomization, regardless of the actual treatment received during the study. For the primary endpoint, non-responder imputation will be used for missing values (including those of randomized subjects who did not receive investigational product).

TABLE 4
Schedule of assessments

| | Screen- | Base- | | | | | | | | |
|---|---|---|---|---|---|---|---|---|---|---|
| | ing | line | | | | | | | | |
| | ≦30 | Day | Treatment Period (week) | | | | | | | |
| Study Visit | days | 1 | 4 | 8 | 10 | 12 | 16 | 20 | 22 | 24 |

General and Safety Assessments

| | | | | | | | | | | |
|---|---|---|---|---|---|---|---|---|---|---|
| Informed Consent | X | | | | | | | | | |
| Medical history | X | | | | | | | | | |
| Medication | X | | | | | | | | | |
| history | | | | | | | | | | |
| Physical exam | X^{c} | X^{c} | X^{c} | X^{c} | | X^{c} | X^{c} | X^{c} | | X^{c} |
| Vital signs | X | X | X | X | | X | X | X | | X |
| Adverse events | | X | X | X | X^{b} | X | X | X | X^{b} | X |
| Concomitant | | X | X | X | | X | X | X | | X |
| medications | | | | | | | | | | |
| Dispensation of | | X | X | X | X^{b} | X | X | X | X^{b} | |
| investigational | | | | | | | | | | |
| drug | | | | | | | | | | |
| Diary review | | | X | X | | X | X | X | | X |

Disease Assessments

| | | | | | | | | | | |
|---|---|---|---|---|---|---|---|---|---|---|
| PASI | X | X | X | X | | X | X | X | | X |
| sPGA | | X | X | X | | X | X | X | | X |
| Involved BSA | X | X | X | X | X^{b} | X | X | X | X^{b} | X |

Laboratory Assessments

| | | | | |
|---|---|---|---|---|
| Hematology | X | X | X | X |
| profile | | | | |
| Chemistry profile | X | X | X | X |
| HIV antibody | X | | | |
| HCV antibody | X | | | |
| HBV surface | X | | | |
| antigen | | | | |
| Urinalysis | X | X | X | X |
| Pregnancy test^{a} | X | X | | |
| Purified protein | X | | | |
| derivative | | | | |
| (PPD) test | | | | |

^{a}Pregnancy test to be performed for all women except those confirmed surgically sterile or at least three years postmenopausal. Screening test will use serum, and baseline test will use urine.
^{b}Week 10 and 12 are clobestasol spray dispensing visits. At the physician and/or coordinators discretion, the subject may be reevaluted to verify appropriate use of clobetasol spray.
^{c}Screening physical exam at screening will be a full physical exam; subsequent exams will be interim exams to measure weight and monitor for any changes.

The patients evaluated for safety endpoints such as adverse events will include all randomized subjects who received at least one dose of the investigational product during the study, with the analysis based on the actual treatment received.

### Example 2

The study described below will compare (1) the safety and efficacy of etanercept therapy combined with a topical foam containing clobetasol propionate (0.05%; OLUX® or OLUX-E® foam) with (2) the safety and efficacy of systemic etanercept treatment in subjects with moderate to severe plaque psoriasis. Efficacy will be measured in a number of ways including the following: the proportion of subjects achieving PASI 50, 75, and 90 at week 12; the proportion of subjects achieving a sPGA score of clear or almost clear at week 12; patient satisfaction with treatment at week 12; percent PASI improvement over baseline PASI score at week 12; incidence and event rates of adverse events; and various laboratory assessment. Additional measurements of efficacy will include assessment of the following: proportions of patients achieving PASI 100 at all time points; improvement from baseline in involved body surface area (BSA) at all time points; improvement in PASI score at all time points; proportion of subjects achieving PASI 50, 75, or 90 or sPGA scores of clear or almost clear at all time points; improvement in Dermatology Life Quality Index (DLQI) and patient satisfaction at all time points; and effect of treatment on C-reactive protein (CRP).

Study Design

This is a randomized, double blind, placebo controlled study to evaluate the safety and efficacy of combining short courses of a topical foam containing clobetasol propionate (0.05%) with systemic etanercept as compared to the safety and efficacy of etanercept monotherapy in patients with moderate to severe plaque psoriasis, as measured by the proportion of subjects achieving a PASI 75 at week 12.

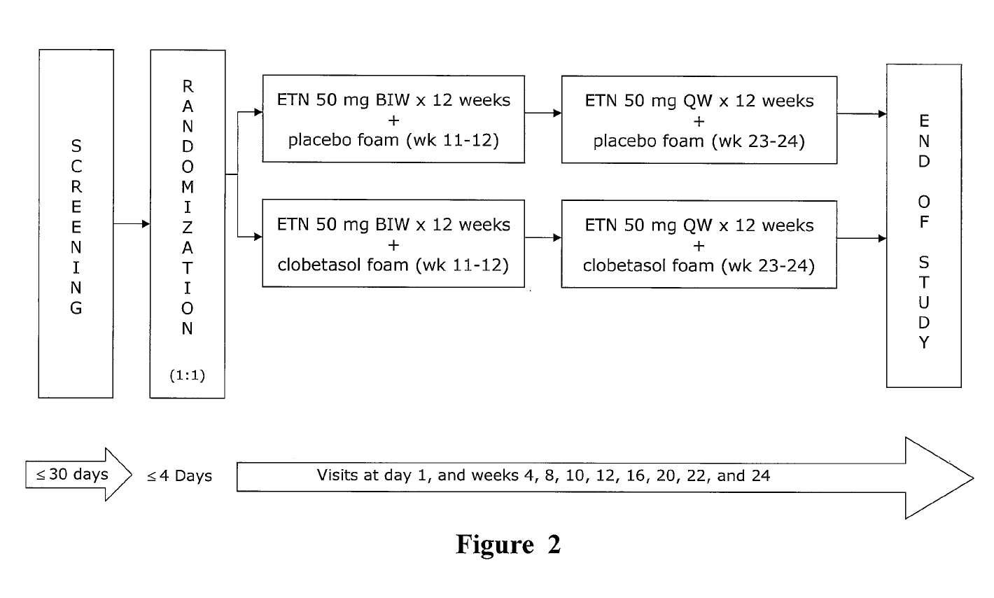

After completing all screening assessments and meeting all eligibility criteria, subjects will be randomized in a 1:1 ratio to receive 12 weeks of etanercept at a dose of 50 mg twice a week with an up to 2-week course of twice-daily clobetasol propionate foam (0.05% foam to be used as determined by a healthcare professional during weeks 11 to 12) or 12 weeks of etanercept at a dose of 50 mg twice a week and an up to 2-week course of twice-daily placebo foam (foam to be used as determined by a healthcare professional during weeks 11 to 12). For the second 12 weeks, subjects will receive etanercept at a dose of 50 mg once a week with an up to 2-week course of twice-daily clobetasol propionate foam or twice-daily placebo foam (foam to be used as determined by a healthcare professional during weeks 23 to 24). There will be no re-randomization. This study design is diagrammed in FIG. 2.

The primary efficacy endpoint will be the proportion of subject to achieve a PASI 75 at week 12. Secondary endpoints include the following: (1) proportion of subjects achieving an sPGA of clear or almost clear at week 12; (2) proportion of subjects achieving a PASI 50 or 90 at week 12; (3) patient satisfaction at week 12; (4) percent PASI improvement from baseline at week 12; (5) incidence and event rates of adverse events; and (6) various laboratory assessments.

Inclusion and exclusion criteria shown in Tables 5 and 6 below will be applied in screening patients for the study.

TABLE 5
Inclusion Criteria

Inclusion Criteria
Subject is capable of understanding and giving written, voluntary informed consent before study
screening.
Subject is a male or female ≧18 years of age at time of screening.
Subject has had stable moderate to severe plaque psoriasis for at least 6 months (e.g., no morphology
changes or significant flares of disease activity in the opinion of the investigator).
Subject has involved BSA ≧10% and PASI ≧10 at screening and at baseline.
Subject has failed at least one topical corticosteroid therapy in the opinion of the investigator
(includes subjects who currently have an involved BSA ≧10% and PASI ≧10 despite current use of a
topical corticosteroid therapy).
Subject is a candidate for systemic therapy or phototherapy in the opinion of the investigator.
Subject has a negative test for hepatitis B virus (HBV) surface antigen and hepatitis C virus (HCV)
antibody.
Subject has a negative purified protein derivative (PPD) test within 30 days prior to the first
etanercept dose. Tuberculin skin tests should be considered positive when they have ≧5 mm of
induration at 48 to 72 hours after test is placed. Subjects with a positive tuberculin skin test (if less
than 15 mm of induration) are allowed if they have a history of Bacillus Calmette-Guerin vaccination
with a negative Quantiferon test in the past year, no symptoms per tuberculosis worksheet, and a
negative chest X-ray.
Subject has a negative serum pregnancy test within 28 days before initiating etanercept for female
subjects (except those at least 3 years postmenopausal or confirmed surgically sterile) and a negative
urine pregnancy test at baseline.
Subject or designee must have the ability to inject etanercept subcutaneously.

TABLE 6
Exclusion Criteria

Exclusion Criteria
Subject has active guttate, erythrodermic, or pustular psoriasis at the time of the screening visit.
Subject has evidence of skin conditions at the time of the screening visit (eg, eczema) that would
interfere with evaluations of the effect of investigational drugs on psoriasis.
Subject diagnosed with medication-induced or medication-exacerbated psoriasis.
Subject has concurrent medical conditions, including: (1) type 1 diabetes; (2) poorly controlled type
2 diabetes (Hemoglobin A1c >8.5); (3) symptomatic heart failure (New York Heart Association
class II, III, or IV); (4) myocardial infarction within the last year; (5) current or history of unstable
angina pectoris within the last year; (6) uncontrolled hypertension as defined by resting blood
pressure >150/90 mmHg prior to randomization (confirmed by a repeat assessment); (7) severe
chronic pulmonary disease (eg, requiring oxygen therapy); (8) major chronic inflammatory disease or
connective tissue disease other than psoriasis and/or psoriatic arthritis; (9) multiple sclerosis or any
other demyelinating disease; (10) active malignancy (including evidence of cutaneous basal or
squamous cell carcinoma or melanoma, or history of cancer (other than fully resected and surgically
cured cutaneous basal cell and squamous cell carcinoma) within 5 years before the first etanercept
dose; if malignancy occurred more than 5 years ago, documentation of disease-free state since
treatment is required); (11) known immunodeficiency syndromes including HIV; (12) alcoholic
hepatitis; or (13) any condition that, in the opinion of the investigator, might cause this study to be
detrimental to the subject.
Subject has any active Common Toxicity Criteria (CTC) grade 2 (localized infection; requiring local
intervention) or higher infection (including chronic or localized infections) within 30 days prior to
screening, at screening, or during screening period prior to first IP dose.
Hypersensitivity to clobetasol propionate, to other corticosteroids, or to any ingredient in the Olux-E ®
preparation.
Subject has any condition that could, in the opinion of the investigator, compromise the subject's
ability to give written consent and/or comply with the study procedures, such as a history of substance
abuse or a psychiatric condition.
Subject has laboratory abnormalities at screening, including: hemoglobin <11 g/dL; platelet
count <125,000/mm³; white blood cell count <3,000 cells/mm³; serum total bilirubin ≧1.5 mg/dL;
aspartate aminotransferase (AST) and/or alanine aminotransferase (ALT) ≧ 1.5 × the upper limit of
normal; creatinine clearance < 50 mL/min (Cockroft-Gault formula, calculated value to be provided
to sites); any other laboratory abnormality, which, in the opinion of the investigator, will prevent the
subject from completing the study or will interfere with the interpretation of the study results.
Subject has used any of the following therapies within 14 days of IP initiation: ultraviolet light B
(UVB) therapy; cyclosporine or calcineurin inhibitors; topical vitamin A or D analog preparations;
or class III through VII topical steroids.
Subject has used any of the following therapies within 28 days of IP initiation: intravenous or oral
calcineurin inhibitors; ultraviolet Light A (UVA) therapy; psoralen plus ultraviolet A radiation
(PUVA); oral retinoids; class I or II topical steroids; anthralin; other systemic psoriasis therapy
(eg, cyclosporine), including oral or parenteral corticosteroids; cyclophosphamide; sulfasalazine; or
methotrexate.
Subject has used one or more biologic therapies (other than interleukin (IL)12/IL23 inhibitors) within
3 months of IP initiation.
Subjects with prior use of an anti-TNF agent if the subject discontinued for lack of efficacy or a
clinically significant adverse event (e.g., serious infection, neurologic event, malignancy, hematologic
event, or any other adverse event that the investigator feels might cause this study to be detrimental to
the subject).
Subject has used an IL-12/IL-23 inhibitor within 6 months of IP initiation.
Subject has ever used efalizumab (Raptiva ®).
Subject is undergoing another investigational procedure.
Subject currently is enrolled in or has not yet completed at least 30 days or 5 half-lives (if applicable;
whichever is longer) since ending other investigational device or drug study(s), or subject is receiving
other investigational agent(s).
Subject is pregnant or breast feeding, or planning to become pregnant within 6 weeks after the end of
treatment.
Female subject is not willing to use highly effective contraception during treatment and for 6 weeks
after the end of treatment.
Male subject is not willing to use highly effective contraception during treatment and for 16 weeks
after the end of treatment.
Male subject (including vasectomised males) with a pregnant female partner is not willing to use
effective methods during treatment and for 16 weeks after the end of treatment to ensure that an
unborn child is not exposed to investigational drugs via semen.

Subjects will be randomized 1:1 to receive etanercept and clobetasol propionate foam (0.05%) or etanercept and a foam placebo. Subjects will be randomized at baseline to etanercept monotherapy with short courses of placebo foam or etanercept monotherapy with short courses of clobetasol propionate foam with a 1:1 ratio. Subjects in the etanercept and placebo foam group will receive etanercept 50 mg twice weekly for 12 weeks along with placebo foam twice per day as needed to clear (foam to be used as determined by the physician during weeks 11 to 12 only), and then step down to etanercept 50 mg once weekly with placebo foam twice per day (foam to be used as determined by the physician during weeks 23 to 24 only) for an additional 12 weeks (a total of 24 weeks). Alternatively, subjects in the etanercept and clobetasol propionate group will receive etanercept 50 mg twice weekly for 12 weeks along with clobetasol propionate foam twice per day as needed to clear (foam to be used as determined by the physician during weeks 11 to 12 only), and then step down to etanercept 50 mg once weekly with clobetasol propionate foam twice per day (foam to be used as determined by the physician during weeks 23 to 24 only) for an additional 12 weeks (a total of 24 weeks).

Randomization will be stratified with respect to body mass index (BMI; BMI<35 kilograms or BMI>35 kilograms), and previous anti-TNF exposure (exposed to any anti-TNF therapies prior to this study or anti-TNF-naïve). In order to achieve balance within different BMI and anti-TNF exposure strata, randomization will be carried out within each of the 4 strata.

Etanercept dosing in both arms will follow the recommended label dosing for patients with psoriasis, that is, 50 mg twice weekly for 12 weeks followed by 50 mg once weekly (for the additional 12 weeks). Each dose of subcutaneous etanercept will consist of the complete contents of 1 syringe. The injection site should be rotated with each dose. During the first 12 weeks of the study, subjects will receive 2 doses of subcutaneous etanercept per week (e.g., on Monday and Thursday). During the second 12 weeks of the study, subjects will receive 1 dose of subcutaneous etanercept per week (scheduled approximately 7 days apart). Throughout the entire trial, administration of subcutaneous etanercept should occur on the scheduled day; however, if unavoidable, it may be given earlier or later as long as the dose is not within 2 days of the next scheduled dose. If the dosing window is missed, that dose should be skipped. Subsequent doses of subcutaneous IP should resume on the original schedule. If etanercept is to be taken the day of a study visit, it must be taken after the study visit has occurred.

Topical clobetasol propionate foam or placebo foam will be applied twice per day, as needed to clear, during two up-to-2-week periods of the trial (foam to be used as determined by the physician during weeks 11 to 12 and weeks 23 to 24). Topical clobetasol propionate foam or placebo foam therapy should be discontinued when control has been achieved. No more than 50 grams or 21 capfuls per week of clobetasol propionate foam or placebo foam is allowed. Use of clobetasol propionate foam or placebo foam on the face or the groin, axillae, or other intertriginous areas is not allowed. Throughout the study, investigators may prescribe any concomitant medications or treatments deemed necessary to provide adequate supportive care except for the following: psoralen plus ultraviolet A (PUVA) therapy; ultraviolet A (UVA) therapy; ultraviolet B (UVB) therapy; cyclosporine; systemically administered calcineurin inhibitors; methotrexate; sulfasalazine; azathioprine; cyclophosphamide; thioguanine; oral retinoids; hydroxyurea; fumarates; any biologic response modifier, including but not limited to efalizumab, alefacept, anakinra, adalimumab, infliximab, and ustekinumab or other IL-12/IL-23 inhibitor; topical steroids; topical vitamin A or D analog preparations; anthralin; live vaccines (e.g., MMR, Varicella, or intranasal flu); oral or parenteral corticosteroids including intramuscular or intraarticular administration (the use of otic, nasal, or inhaled corticosteroids within recommended doses is allowed); or any investigational therapy other than etanercept or clobetasol propionate foam. Clobetasol propionate foam is permitted to be used only as described in the protocol.

Patients will be assessed for various medical parameters throughout the protocol as detailed in Table 7.

TABLE 7
Schedule of assessments

| | Screen- | Base- | | | | | | | | |
|---|---|---|---|---|---|---|---|---|---|---|
| | ing | line | | | | | | | | |
| | ≦30 | Day | Treatment Period (week) | | | | | | | |
| Study Visit | days | 1 | 4 | 8 | 10 | 12 | 16 | 20 | 22 | 24 |

General and Safety Assessments

| | | | | | | | | | | |
|---|---|---|---|---|---|---|---|---|---|---|
| Informed Consent | X | | | | | | | | | |
| General and | | | | | | | | | | |
| Safety | | | | | | | | | | |
| Assessments | | | | | | | | | | |
| Informed Consent | X | | | | | | | | | |
| Medical/ | X | | | | | | | | | |
| medication | | | | | | | | | | |
| history | | | | | | | | | | |
| Physical exam | X^{a} | X^{a} | X^{a} | X^{a} | | X^{a} | X^{a} | X^{a} | | X^{a} |
| Vital signs | X | X | X | X | | X | X | X | | X |
| Adverse events | X^{b} | X | X | X | X | X | X | X | X | X |
| Concomitant | | X | X | X | | X | X | X | | X |
| medications | | | | | | | | | | |
| Subcutaneous | | X | X | X | | X | X | X | | |
| etanercept | | | | | | | | | | |
| dispensation | | | | | | | | | | |
| Topical clobetasol | | | | | X | | | | X | |
| propionate | | | | | | | | | | |
| dispensation | | | | | | | | | | |
| Diary review | | | X | X | | X | X | X | | X |

Disease Assessments

| | | | | | | | | | | |
|---|---|---|---|---|---|---|---|---|---|---|
| PASI | X | X | X | X | X | X | X | X | X | X |
| sPGA | | X | X | X | X | X | X | X | X | X |
| Involved BSA | X | X | X | X | X | X | X | X | X | X |

Laboratory Assessments

| | | | | |
|---|---|---|---|---|
| Hematology/ | X | X | X | X |
| chemistry/ | | | | |
| urinalysis | | | | |
| CRP | | X | X | X |
| HCV antibody, | X | | | |
| HBV | | | | |
| surface antigen | | | | |
| Pregnancy test^{c} | X | X | | |
| Purified protein | X | | | |
| derivative | | | | |
| (PPD) test | | | | |
| Chest radiograph^{d} | X | | | |
| Latent | X | | | |
| tuberculosis | | | | |
| test^{d} | | | | |

Subject Completed Assessments

| | | | | | |
|---|---|---|---|---|---|
| Patient | X | X | X | X | X |
| Assessment | | | | | |
| of treatment | | | | | |
| satisfaction | | | | | |
| DLQ1 | X | X | X | X | X |

Photography (only for consenting subjects)

| | | | |
|---|---|---|---|
| Photography | X | X | X |

^{a}Physical exam at screening will be a full physical exam. Subsequent exams will measure weight and monitor for any changes.
^{b}Serious adverse events to be reported from signing of informed consent through 30 days after last dose. Adverse events to be reported from randomization to last study visit.
^{c}Pregnancy test to be performed for all women except those confirmed surgically sterile or at least three years postmenopausal. Screening test will use serum, and baseline test will use urine.
^{d}These tests are only done if the PPD test is positive and the subject has a history of Bacillus of Calmette and Guerin (BCG) vaccination.

1-38. (canceled)

39. A method for treating a patient having psoriasis, comprising:
(a) selecting a patient who has
(i) a PASI score of at least about 10 and at least about 10% of his/her body surface area (BSA) affected by psoriasis and/or
(ii) a sPGA score of >2;
(b) administering etanercept to the patient at a dose of about 50 milligrams twice per week for a first time period of about 12 weeks and then administering etanercept to the patient at a dose of about 50 milligrams once per week for an additional time period of about 12 weeks;
(c) administering
(i) an upper midstrength, potent, or superpotent topical preparation containing a glucocorticoid in any one of Classes 1-3 to the patient once during the first and/or the additional time period of (b), wherein the topical preparation is administered at least once per week for a continuous time period of not more than four weeks or
(ii) the topical preparation of (c)(i) intermittently during the first and/or additional time period of (b), wherein the topical preparation is administered on an as-needed basis for one or more continuous time periods of not more than four weeks each, wherein each continuous time period in which the topical preparation is administered is separated by an intervening continuous time period in which the topical preparation is not administered, which is at least as long as the preceding continuous time period during which the topical preparation was administered; and
(d) assessing the PASI and/or the sPGA score of the patient at the end of the first time period of (b);
wherein the treatment is therapeutically effective.

40. The method of claim 39,
wherein the patient achieves a PASI 75 by the end of the first time period of (b) and
wherein the patient maintains the PASI 75 at the end of the additional time period of (b).

41. The method of claim 39, wherein the topical preparation is administered on an as-needed basis for a continuous time period of not more than two weeks during the first and/or the additional time period of (b).

42. The method of claim 39, wherein
the topical preparation of (c)(i) is administered intermittently during the first and/or additional period of (b),
the topical preparation is administered on an as-needed basis for one or more continuous time periods of not more than two weeks each, and
each continuous time period in which the topical preparation is administered is separated by an intervening continuous time period in which the topical preparation is not administered at least as long as the preceding continuous time period during which the topical preparation was administered.

43. The method of claim 39, wherein etanercept administration is continued for at least about six months following the additional time period of (b) at a dose of about 50 milligrams per week, and wherein the patient achieves a PASI 75 at the end of the six months.

44. The method of claim 39, wherein etanercept administration is continued for at least about a year following the additional time period of (b) at a dose of about 50 milligrams per week, and wherein the patient maintains a PASI 75 at the end of the year.

45. The method of claim 39, wherein the patient has an sPGA score of clear or almost clear by the end of the first time period of (b) and/or the patient has an sPGA score of clear or almost clear by the end of the additional time period of (b).

46. The method of claim 41, wherein the patient has an sPGA score of clear or almost clear by the end of the first time period of (b) and/or the patient has an sPGA score of clear or almost clear by the end of the additional time period of (b).

47. The method of claim 44,
wherein the topical preparation is administered during one continuous time period or during intermittent continuous time periods during the year,
wherein each continuous time period in which the topical preparation is administered is not more than four weeks, and
wherein each continuous time period in which the topical preparation is administered is separated by an intervening continuous time period in which the topical preparation is not administered at least as long as the preceding continuous time period during which the topical preparation was administered.

48. The method of claim 47, wherein each continuous time period in which the topical preparation is administered is not more than two weeks.

49. The method of claim 43, wherein the topical preparation is administered during not more than three continuous time periods during the six months.

50. The method of claim 44, wherein the topical preparation is administered during not more than six continuous time periods during the year.

51. The method of claim 39, wherein the patient achieves a PASI 90 by 12 weeks after the beginning of the first time period of (b) and wherein the patient maintains the PASI 90 at the end of the additional time period of (b).

52. The method of claim 39, wherein the topical preparation is a potent or superpotent preparation.

53. The method of claim 52, wherein the glucocorticoid in the topical preparation is betamethasone dipropionate.

54. The method of claim 53, wherein the topical preparation further comprises calcipotriene.

55. The method of claim 52, wherein the glucocorticoid in the topical preparation is clobetasol propionate.

56. The method of claim 55, wherein the topical preparation is a spray.

57. The method of claim 55, wherein the topical preparation is a foam.

58. The method of claim 41, wherein the topical preparation is a potent or superpotent preparation.

59. The method of claim 58, wherein the glucocorticoid in the topical preparation is clobetasol propionate.

60. A method for treating a psoriasis patient who is receiving etanercept comprising
continuing treatment with etanercept,
initiating treatment with an upper midstrength, potent, or superpotent topical preparation containing a glucocorticoid,
continuing treatment with the topical preparation containing the glucocorticoid for a first, time period of not more than 4 weeks, and
then discontinuing treatment with the topical preparation containing the glucocorticoid for a second time period of at least as long as the first time period.

61. The method of claim 60, wherein the patient continues the treatment with etanercept for at least about a year.

62. The method of claim 60, wherein the patient maintains a sPGA score of 0 or 1 for the year during which treatment is continued.

63. The method of claim 60, wherein the glucocorticoid is clobetasol propionate.

64. The method of claim 60, wherein the glucocorticoid is betamethasone dipropionate.

65. A method for treating a patient having psoriasis and haying a PASI score of at least about 10 and an affected BSA of at least about 10% and/or a PGA score of >2 comprising
administering to the patient etanercept at a dosage of about 50 milligrams twice per week for a first time period of at least about 12 weeks,
administering to the patient a topical preparation comprising clobetasol propionate or betamethasone dipropionate plus calcipotriene as needed for at most 4 weeks during the first time period, wherein the patient achieves a PASI 75 at the end of the first time period;
administering etanercept for an additional time period of at least about 12 weeks thereafter at a dosage of about 50 milligrams per week, wherein the patient maintains a PASI 75 at the end of the additional time period.

66. A method for treating psoriasis comprising
(a) selecting a patient who has the following characteristics:
(i) the patient has a sPGA score of >2 and/or a PASI score of at least 10 with at least 10% of the BSA affected;
(ii) the patient does not have a condition selected from the group consisting of: congestive heart failure; severe pulmonary disease; systemic lupus erythematosus; multiple sclerosis or any other demyelinating disease; pregnancy; and breast feeding;
(iii) the patient does not exhibit any of the following laboratory abnormalities: hemoglobin<11 g/dL; platelet count<125,000/mm³; white blood cell count<3000 cells/mm³; AST and/or ALT≧1.5× the upper limit of normal; and
(iv) a topical preparation containing a glucocorticoid has previously been used to treat the patient's psoriasis, wherein the patient did not achieve a sPGA of clear or almost clear during this treatment;
(b) administering to the patient etanercept;
(c) intermittently administering to the patient an upper midstrength, potent, or superpotent topical preparation containing a glucocorticoid on an as-needed basis.

67. A method for treating plaque psoriasis comprising
(a) selecting a patient who has the following characteristics:
(i) the patient has a sPGA score of >2 and/or a PASI score of at least 10 with at least 10% of the BSA affected;
(ii) the patient does not have a condition selected from the group consisting of: congestive heart failure; severe chronic pulmonary disease; systemic lupus erythematosus; multiple sclerosis or any other demyelinating disease; pregnancy; and breast feeding; type I diabetes; poorly controlled type 2 diabetes (Hemoglobin Alc>8.5); symptomatic heart failure (New York Heart Association class II, III, or IV); myocardial infarction within the last year; current or history of unstable angina pectoris within the last year; uncontrolled hypertension as defined by resting blood pressure>150/90 mmHg prior to randomization; major chronic inflammatory disease or connective tissue disease other than psoriasis and/or psoriatic arthritis; active malignancy within 5 years before the first etanercept dose; known immunodeficiency syndromes including HIV; or alcoholic hepatitis;
(iii) the patient does not exhibit any of the following laboratory abnormalities: hemoglobin<11 g/dL; platelet count<125,000/mm³; white blood cell count<3000 cells/mm³; aspartate aminotransferase (AST) and/or alanine aminotransferase (ALT)≧1.5× the upper limit of normal; serum total bilirubin≧1.5 milligrams/dL; and
(iv) the patient has a negative test for hepatitis B virus (HBV) surface antigen and hepatitis C virus (HCV) antibody;
(v) a topical preparation containing a glucocorticoid has previously been used to treat the patient's psoriasis, wherein the patient did not achieve a sPGA of clear or almost clear during this treatment;
(b) administering to the patient etanercept;
(c) intermittently administering to the patient an upper midstrength, potent, or superpotent topical preparation containing a glucocorticoid on an as-needed basis.
